(19) United States
(12) Patent Application Publication
(10) Pub. No.: US 2016/0069878 A1
(43) Pub. Date: Mar. 10, 2016

(54) SEMI-AUTOMATED WHOLE BLOOD IMMUNO-POTENCY ASSAY

(71) Applicant: NATIONAL UNIVERSITY OF IRELAND, GALWAY, Galway (IE)

(72) Inventors: Rhodri Ceredig, Galway (IE); Andreia Ribeiro, Galway (IE)

(21) Appl. No.: 14/787,367

(22) PCT Filed: May 7, 2014
(86) PCT No.: PCT/EP14/59397

§ 371 Date: Oct. 27, 2015

(30) Foreign Application Priority Data
May 8, 2013 (GB) 1308271.4

(51) Int. Cl. G01N 33/569; G01N 33/50

(52) U.S. Cl. CPC G01N 33/56972; G01N 33/5094; G01N 2500/10

(57) ABSTRACT

The present invention relates to a rapid, semi-automated whole blood assay to quantify the potency of cultured stem cells and biologicals in inhibiting monocyte and inducing T cell activation. Such an assay allows the quantification of the anti-inflammatory potency of therapeutic stem cell products for individual patients.

### FIELD OF THE INVENTION

The present invention relates to a rapid, semi-automated whole blood assay to quantify the potency of cultured stem cells and biologicals in inhibiting monocyte and inducing T cell activation. Such an assay allows the quantification of the anti-inflammatory potency of therapeutic stem cell products for individual patients.

### BACKGROUND TO THE INVENTION

Stem Cell Biology has progressed into the clinical and commercial arenas for a wide range of disease targets including gastrointestinal, immunological and heart diseases. It is now clear that the more immediately applicable therapeutic property of cultured stem cells is their capacity to modulate the behaviour of cells around them via contact-dependent and soluble mediators. This paracrine, or “trophic”, effect includes potent anti-inflammatory and immune suppressive properties that form the basis for the use of cultured stem cell products to treat common inflammatory and autoimmune conditions such as myocardial infarction, limb ischemia, skin ulceration, osteoarthritis, inflammatory bowel disease (IBD), multiple sclerosis and rheumatoid arthritis. The Mesenchymal Stem (or Stromal) Cell (MSC) has been the predominant focus for translational researchers and healthcare companies. This preference has arisen from the facts that MSCs can be cultured from a variety of accessible tissues (bone marrow, fat, placenta, umbilical cord, gingival), are amenable to large-scale culture expansion and have been convincingly shown to mediate potent anti-inflammatory effects in vitro and in vivo.

Commercial development in the Stem Cell Sector has increased dramatically in the past 6 years and therapeutic stem cells, particularly off-the-shelf, culture-expanded MSCs, now represent commercial products with the potential for significant variability in efficacy. Furthermore, the requirement for potency/efficacy assays will, almost certainly become a requirement for approval of stem cell therapeutic products. This is because the potency of human culture-expanded MSC for monocyte and T-cell suppression depends on tissue source, donor and passage number and varies for individual subjects. Thus, a clear need exists for healthcare providers to select patients with the greatest risk-benefit ratio for stem cell therapy as well as to select an optimal product for each patient.

Monocytes develop from myelo-monocytic stem cells in the bone marrow. They then go into blood, where they circulate for a few days and then migrate into the tissues. In the tissue they further mature into macrophages. Monocytes play important roles in the inflammatory response. They are positive for CD13, CD14, CD15, CD16, CD64, CD11 (b and c) and HLA-DR.

T cells are a subset of lymphocytes that play a large role in the immune response and are at the core of adaptive immunity. T cells may be detrimental in diseases in which inflammation is linked to tissue destruction. They are positive for CD2, CD3, CD5, CD7, TCR CD45RA (naïve) and CD45Ro (memory).

The therapeutic properties of MSCs for inflammatory and immune-mediated disease are linked to their modulatory effects on monocyte/macrophages and T-cell activation. The best-documented anti-inflammatory effects of MSCs in such diseases are “re-programming” of monocyte/macrophages to produce anti-inflammatory cytokines such as interleukin 10 (IL-10) and the suppression of the activation of T-cell subsets responsible for production of pro-inflammatory cytokines such as interferon gamma (IFNγ) and interleukin 17 (IL-17). No good technique for tailoring individual MSC products to the patients most likely to benefit from them, has been generated. There is clearly a need to optimise therapy to individual patients—an approach to healthcare that is often referred to as “personalized medicine”.

Jiao et al Methods Mol Biol 2011, 677, 221-31 describes an assay for the potency of MSC-CM and MSC-ly based on IL-10.

### OBJECT OF THE INVENTION

It is thus an object of the present invention to develop an automated, flow-cytometry-based human whole-blood assay of MSC anti-inflammatory potency. A further object is to develop an assay of MSC anti-inflammatory potency which can produce results in about 24 hours or less. The assay should also be reliable. Such an assay will take the potential recipient's blood cells and determine which batch of potential donor allogeneic MSC is the most potent at inhibiting the patient's monocytes and T cells.

Thus a further object of the invention is to develop an assay to quantify MSC re-programming of stimulated human monocytes. Another object is to develop an assay to quantify MSC suppression of human T-cell activation. In particular it is an object to provide an assay for determining the potency of human Bone Marrow Mesenchymal stromal Cells (hBM MSC).

A further object of the invention is to provide an assay that can be used to identify immunomodulatory compounds. This can be done by titrating compounds into 96 well plates and carrying out the blood activation in the presence of graded amounts of compounds and determining which compounds have an immunomodulatory effect on the blood cells.

### SUMMARY OF THE INVENTION

According to the present invention there is provided whole blood immuno-potency assay comprising the steps:

(1) collecting a blood sample from a patient in a heparinised tube'

(2) diluting the blood sample to between ⅕ and 1/20 in culture medium, and then adding LPS, Brefeldin A to the blood sample,

(3) adding a mix of antibodies directed against surface molecules on the cells in the treated blood sample in the tube,

(4) adding formaldehyde and incubating at room temperature,

(5) Concentrating the cells by centrifugation,

(6) Discarding the supernatant and adding Saponine to the pellet,

(7) adding PE-labelled antibody to the permeabilised cells,

(8) concentrating the cells, and

(9) analysing the cells by flow cytometry.

Preferably the blood in step (2) is diluted 1/10. The treated blood in step (2) may be incubated for between 4 and 36 hours.

The LPS may be added at a concentration of between 0.5 and 20 ng/ml, with a concentration of 2 ng/ml being preferable. Brefeldin A may be added at a concentration of between 0.75 and 3.1 μg/ml, with a concentration of 0.6 μg/ml being preferable.

In step (3) the sample may be incubated for about 10 min in the dark.

The formaldehyde used in step (4) may be Reagent 1 which is part of the Intraprep kit from Beckman Coulter ref. A07803. Following addition of formaldehyde or Reagent 1 the sample may be incubated at room temperature for about 10 min in the dark.

The Saponine used in step (6) may be Reagent 2 which is part of the Intraprep kit from Beckman Coulter ref. A07803. This results in permeabilisation of the cells.

The PE labelled antibody in step (7) may be TNF-α PE or IL-12-PE. The cells may be incubated with the antibody for about 10 mins in the dark.

Following incubation in step (7) the cells may be washed by adding 400μ phosphate-buffered saline (PBS) and then the cells may be concentrated by centrifugation.

The washing step may then be repeated and the cells may then be resuspended in PBS.

The blood sample may be incubated for 6 hours for the determination of TNF-α and for 24 hours for the determination of IL-12. The incubation temperature may be 37° C. in 5% CO₂.

The fluorescently-labelled antibodies to surface molecules on monocytes may be selected from CD45 and CD14. To study T cells, a combination of antibodies to CD3 and CD8 may be used.

The blood sample may be incubated with the antibodies for 10 min, in the dark, at room temperature. Incubation with of Reagent 1 may be for 10 min, in the dark, at room temperature.

The cells may be concentrated in step (6) at 1500 rpm for 5 min at 20° C.

The cells may be incubated with TNF-α PE for 15 min, in dark, at room temperature.

Concentration of the cells in step (9) may be at 1500 rpm for 5 min at 20° C.

Flow cytometry allows identification or gating of monocytes by a combination of light scatter and fluorescence signals and intracellular TNF-α or IL-12 measured on gated monocytes.

### BRIEF DESCRIPTION OF THE DRAWINGS

FIG. 1 illustrates intra-cellular staining for TNFα of gated human peripheral blood monocytes stimulated for 6 hr with different doses of LPS. This shows that with increasing LPS dose from 0.5 to 20 ng/ml LPS, a greater proportion of monocytes express intra-cytoplasmic TNFα reaching a maximum at about 5 ng/ml LPS.

FIG. 2 illustrates the effect of adding increasing numbers of allogeneic MSC on the expression of TNFα by monocytes stimulated for 6 hr with 2 ng/ml LPS. AS can be seen, there is an MSC cell dose-dependent inhibition of TNFα expression.

FIG. 3 illustrates the different potencies of different MSC sources on different blood donors.

FIG. 4 illustrates that different blood donors react differently to the different MSCs.

FIG. 5 shows the mean potency with a fixed MSC to monocyte ratio was fixed.

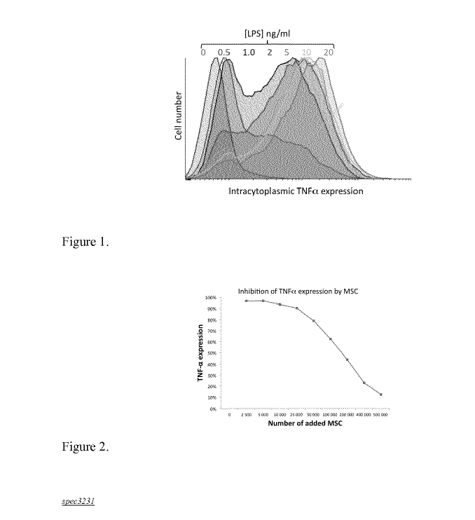

### DETAILED DESCRIPTION OF THE DRAWINGS

The automated assay of the invention allows quantification of the immune-modulatory properties of MSC on both monocyte and T cell activation. Which population of cells that is monitored depends on the combination of fluorescently-labelled monoclonal antibodies used. The protocol adopted has been developed so that it can be run in a fully automated way. For this, initial whole blood cell cultures are set up on a Perkin-Elmer Janus robotic work station using 1 ml culture vessels in a 96 well microtiter plate format. Following a 4 hr culture, cells are transferred to a second 96 well plate for staining and analysis on a B-D Accuri C6 flow cytometer fitted with an automated 96 well plate C6 sample acquisition module. This automated assay format has been successfully run with MSC. In addition to studying the effects of adding cells to monocyte or T cell activation, the assay can also be used to screen the immune-modulatory properties of soluble compounds.

The assay has been developed to have a rapid turnaround, automated assay. It can be carried out on a robot. Results can be obtained within 6 hours. TNFα production is the most rapid cytokine to be released by monocytes and is quicker than IL-10 which takes about 24 hr.

In the method of the invention Brefeldin-A is added to the cells in order to block glycosylation and prevent secretion of cytokines, so no cytokine is found in the supernatant. The advantage of such a strategy id that the frequency of cytokine-secreting cells among defined sub-populations can be directly quantitated.

The assay can also be used to determine T cell activation. Similarly, this assay can be automated and looks at γIFN production by gated CD8+ T cells with a six hour turnaround. Thus the assay of the invention can be used to study the effect of added cells and biologically active compounds on both monocytes and T cells.

### EXAMPLE

### Assay Protocol

### 1. Activation of Cells

Collect blood in a Heparinised tube.

Put 50 ul blood, 1 ng/ml LPS, 0.6 ug/ml Brefeldin A and RPMI-1640 Medium (Sigma # R0883); final volume is 500 ul. For the other blood dilutions, the volume of blood added is changed. This gives a final dilution of blood of 50 ul in 500 ul, or 1/10. Other dilutions (for example 1:20) can be established by for example reducing the volume of blood to 25 ul but keeping the final volume to 500 ul.

LPS source: (InvivoGen # tlrl-3 pelps) we tested other concentrations: 0.5, 2, 5, 10 and 20 (ng/mL)

Brefeldin A (eBioscience # 00-4506-51) final concentration 0.6 μg/ml—we tested (μg/ml): 0.75, 1.12, 1.5, 2.25 and 3.1

The treated blood is then incubated for 6 H for TNF-α and 24 H for IL-12, at 37° C. in 5% CO₂. We also tested 4 H and 8 H of incubation.

### 2. Intracellular Staining (Beckman Coulter IntraPrep Kit # A07803)

1) Add a mix of fluorescently-labelled antibodies directed against surface molecules expressed by monocytes to the treated blood sample in a 96 deep well plate (CD45 PerCpCy5.5 eBioscience # 45-9459-42 and CD14 APC eBioscience # 17-0149-42

2) Incubate for 10 min, in dark, room temperature

3) Add 60 μL of Reagent 1 Incubate 10 min, in dark, at room temperature

4) Add 300 μL PBS 1× and concentrate cells, 1500 rpm, 5 min, 20° C.

5) Discard supernatant

6) Add 50 μL Reagent 2

7) Add diluted (generally about 1/100 final concentration, exact dilution depends on the batch of antibody) TNF-α PE (cat 12-7349 eBioscience). IL-12 is detected after 24 h incubation with IL-12-PE antibody (cat 12-7235 eBioscience). Incubate 15 min, in dark, room temperature

8) Add 600 μL PBS 1× and concentrate cells, 1500 rpm, 5 min, 20° C.

9) Discard supernatant

10) Repeat the wash with 900 μL PBS 1×

11) Resuspend in 60 μL of FACS Buffer

12) Acquire sample in ACCURI flow cytometer. This machine measures light scatter and fluorescence signals on defined subpopulations of cells in a complex mix such as blood. Using flow cytometry allows one to quantitate cytokine production by gated subpopulations of cells such as monocytes or CD8+ T cells.

It is important that the blood is in an anticoagulant. Heparin is preferred as the anticoagulant. Citrate and EDTA chelate calcium and cannot be used.

Optimally the blood needs to be diluted 1/10. We have tested over a dilution range of ½ ⅕, 1/10, 1/20 and 1/30. If there is insufficient dilution, the test doesn't work and with too much dilution there aren't enough cells to analyse.

The LPS concentration range that works is wide (0.1-100 ng/ml). By using about 1 ng/ml, which is on the exponential part of the dose/response curve, the test is sensitive to inhibition by added cells or soluble compounds. With more LPS it's harder to inhibit activation.

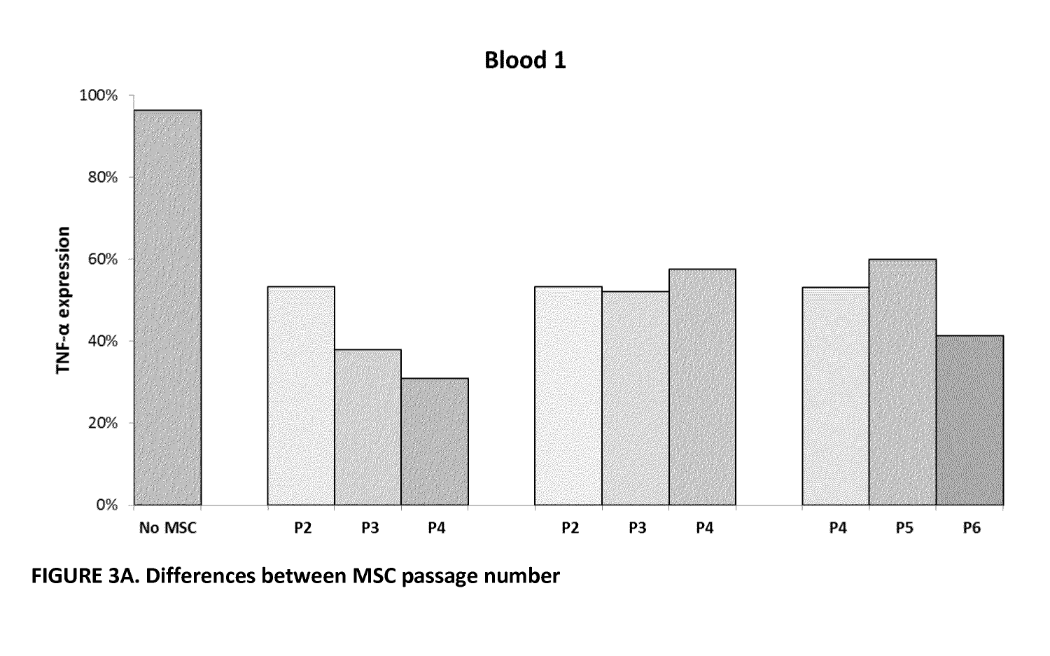

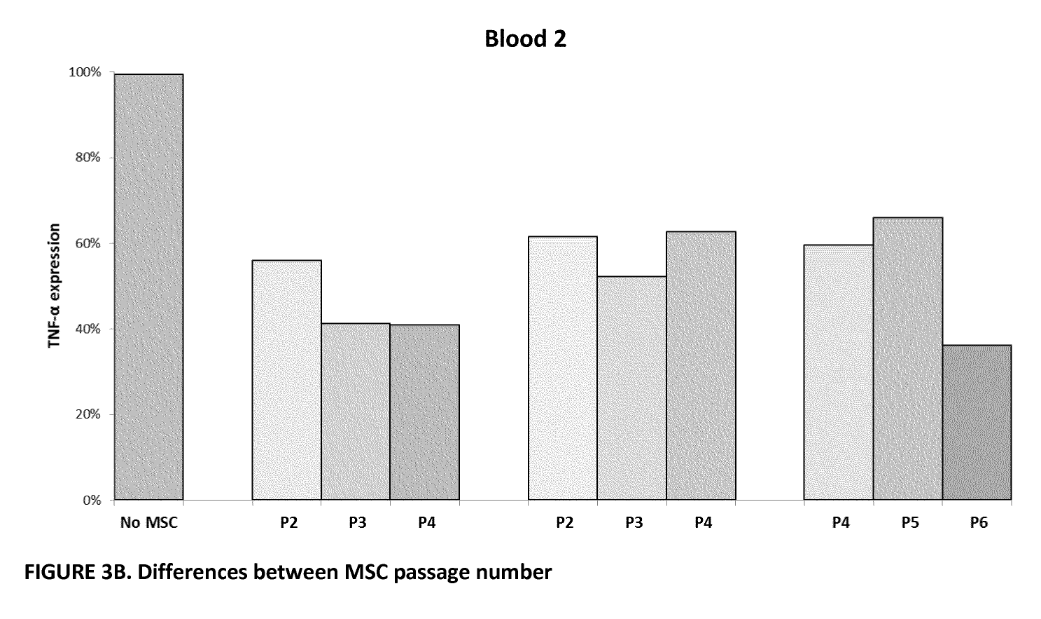

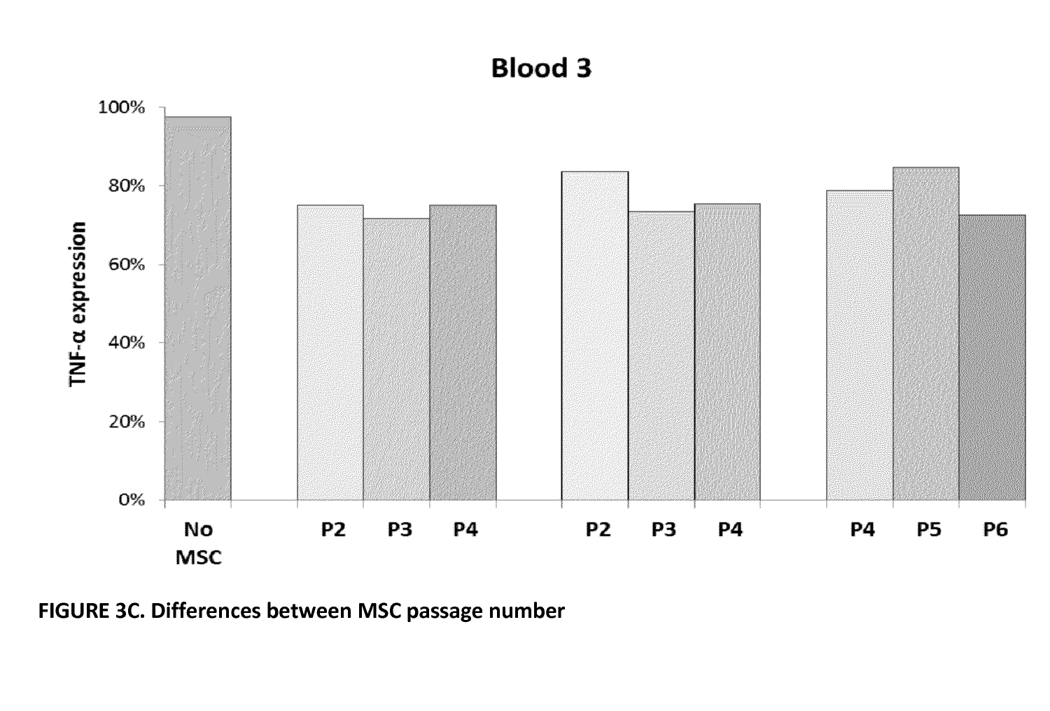

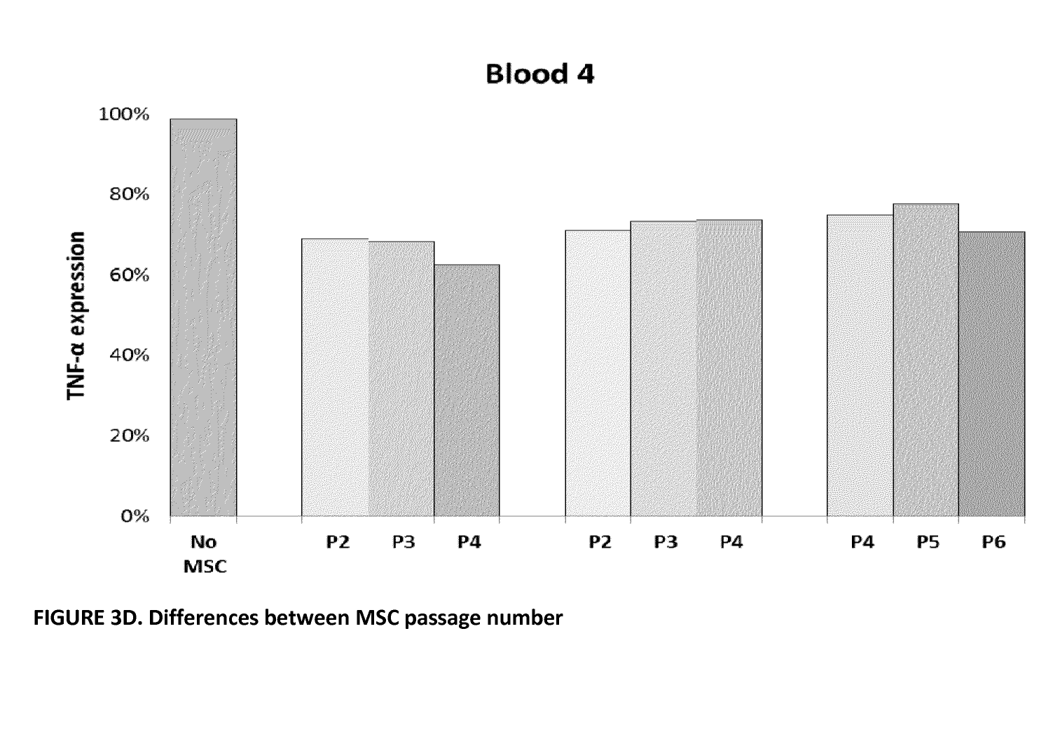

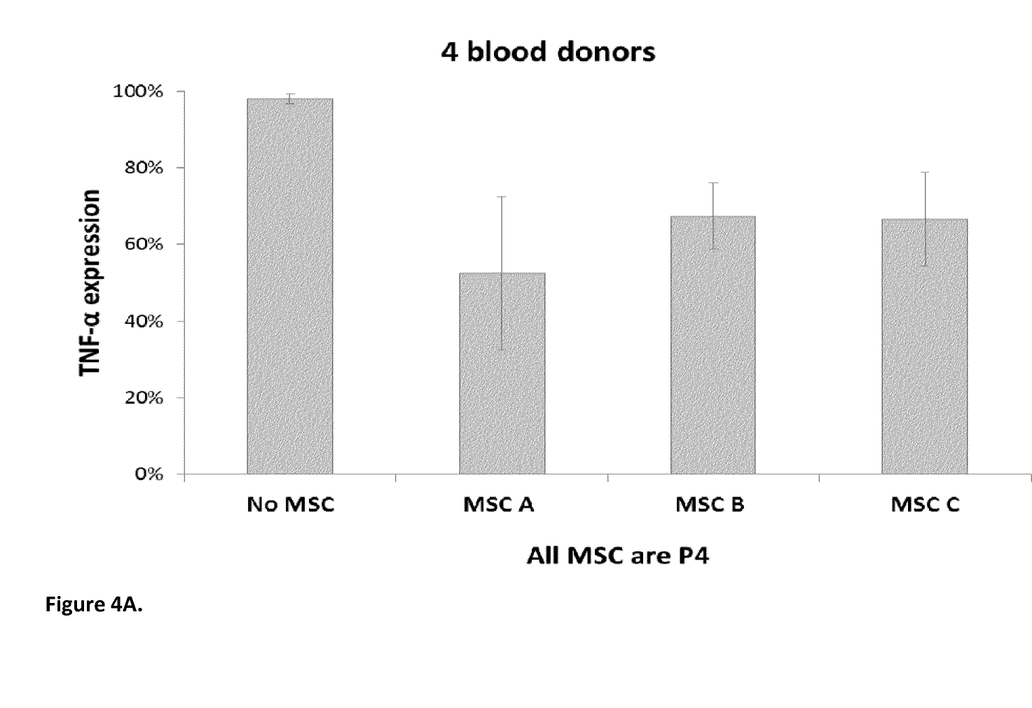

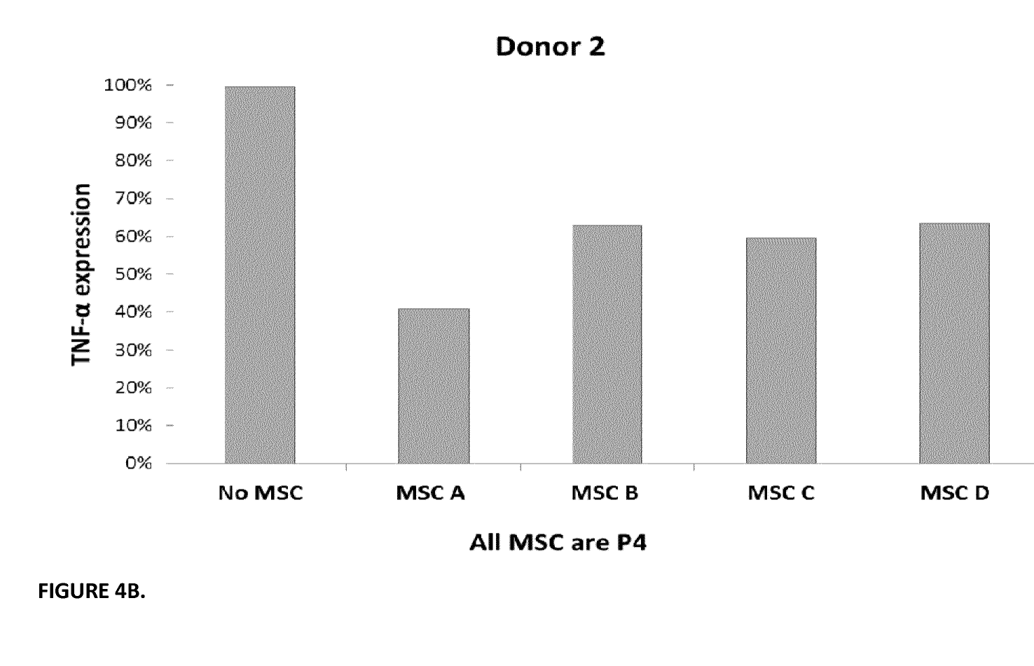

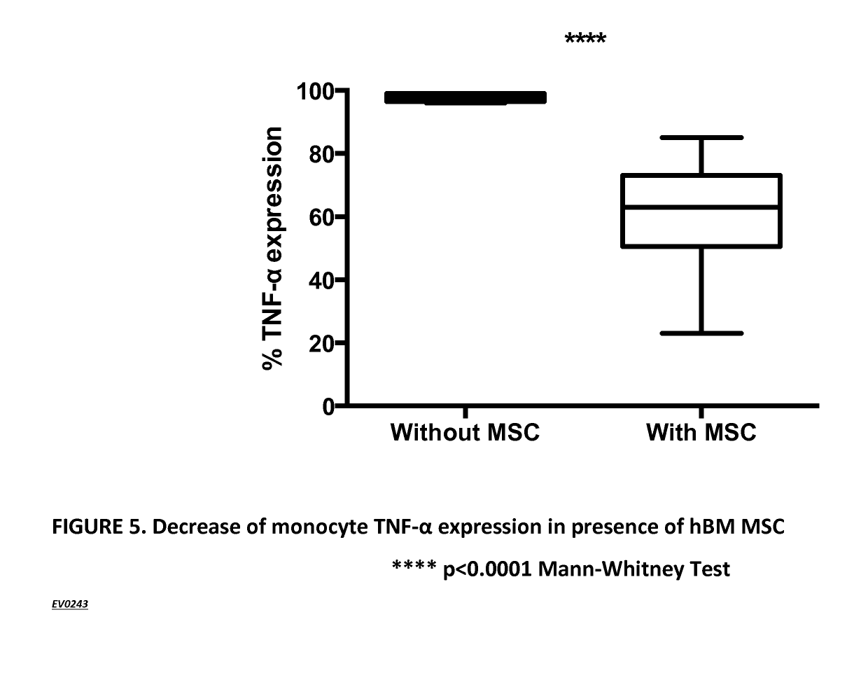

FIG. 3 shows that different MSC sources do seem to have different potency on the various blood donors. For example, MSCA inhibits Blood 1 and 2, but not so much 3 and 4. Thus the assay of the invention allows us to predict how the patient is going to react to the MSC treatment. FIG. 4 shows that different blood donors behave differently to the different MSC, so blood from donor 2 is inhibited by MSC from A, but less so by MSC from B, C and D. Thus the assay allows a comparison of the efficiency of different MSC donors for each patient. FIG. 5 shows the mean potency being measured where the MSC to monocyte ratio was fixed based on “potent” MSCs commercially obtained from Orbsen. This graph shows that there is a decrease of monocyte TNF-α expression in presence of hBM MSC.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A whole blood immuno-potency assay comprising the steps:
(1) collecting a blood sample from a patient in a heparinised tube,
(2) diluting the blood sample to between ⅕ and 1/20 in culture medium, and then adding LPS, Brefeldin A to the blood sample,
(3) adding a mix of antibodies directed against surface molecules on the cells in the treated blood sample in the tube,
(4) adding formaldehyde and incubating at room temperature,
(5) Concentrating the cells by centrifugation,
(6) Discarding the supernatant and adding Saponine to the pellet,
(7) adding PE-labelled antibody to the permeabilised cells,
(8) concentrating the cells, and
(9) analysing the cells by flow cytometry.

2. An assay as claimed in claim 1 wherein the blood in step (2) is diluted 1/10.

3. An assay as claimed in claim 1 or 2 wherein the LPS is added at a concentration of between 0.5 and 20 ng/ml.

4. An assay as claimed in claim 3 wherein the LPS is added at a concentration of, with a concentration of 2 ng/ml.

5. An assay as claimed in any preceding claim wherein Brefeldin A is added at a concentration of between 0.75 and 3.1 μμg/ml.

6. An assay as claimed in claim 5 wherein the Brefeldin A is added at a concentration of 0.6 μg/ml.

7. An assay as claimed in any preceding claim wherein the blood sample in step (2) is diluted 1/10.

8. An assay as claimed in any preceding claim wherein following addition of formaldehyde the sample is incubated at room temperature for about 10 min in the dark.

9. An assay as claimed in any preceding claim wherein the PE labelled antibody in step (7) is selected from may be TNF-α PE or IL-12-PE.

10. An assay as claimed in any preceding claim wherein following incubation in step (7) the cells are washed by adding 400 μl phosphate-buffered saline (PBS) and then the cells are concentrated by centrifugation.

11. An assay as claimed in any preceding claim wherein the fluorescently-labelled antibodies directed against surface molecules on monocytes or T cells are selected from CD45, CD14, CD3 and CD8.

12. A method of identifying novel immunosuppressive compounds comprising analysing the test compound by a method as claimed in any preceding claim.

13. A whole blood immuno-potency assay as claimed in any preceding claim substantially as described herein with reference to the Example or the accompanying drawings.

14. A method of identifying novel immunosuppressive compounds substantially as described herein with reference to the Example or the accompanying drawings.
